(19) United States
(12) Patent Application Publication
(10) Pub. No.: US 2010/0286534 A1
(43) Pub. Date: Nov. 11, 2010

(54) METHODS AND SYSTEMS FOR ASSESSING METABOLIC TRANSITION POINTS

(75) Inventor: Shai Greenberg, Herzelia (IL)

Correspondence Address: MARTIN D. MOYNIHAN d/b/a PRTSI, INC., P.O. BOX 16446, ARLINGTON, VA, 22215, US

(73) Assignee: ALTERNA, LIMITED, Makabim (IL)

(21) Appl. No.: 12/307,087

(22) PCT Filed: Jun. 29, 2007
(86) PCT No.: PCT/IB07/03773

§ 371 Date: Apr. 19, 2010

Related U.S. Application Data
(60) Provisional application No. 60/817,557, filed on Jun. 30, 2006.

(51) Int. Cl. A61B 5/0205

(52) U.S. Cl. 600/484

(57) ABSTRACT

The invention relates to methods and systems for determining one or more metabolic transition points in a subject during a work task. The methods and systems comprise determining the respiration rate (RR) and heart rate (HR) in the subject that is undergoing a work task and calculating the ratio of RR/HR at more than one time point during the work task. The metabolic transition points are identifiable points in time of the RR/HR ratio in the subject that is undergoing a work task.

### CROSS-REFERENCE TO RELATED APPLICATIONS

This application claims priority to U.S. Provisional Application No. 60/817,557, filed 30 Jun. 2006, which is incorporated by reference.

### BACKGROUND OF THE INVENTION

1. Field of the Invention

The invention relates to methods and systems for determining one or more metabolic transition points in a subject during a work task. The methods and systems comprise determining the respiration rate (RR) and heart rate (HR) in the subject that is undergoing the work task and calculating the ratio of RR/HR at more than one time point during the work task. The metabolic transition points are identifiable points in time of the RR/HR ratio in the subject that is undergoing a work task.

2. Background of the Invention

Physical performance requires the integration of the physiological responses of the cardiovascular system and the ventilation systems of the body. To perform physical activities, muscles rely on a pool of adenosine triphosphate (ATP) as a direct source of energy. With the exception of short-time work for which only phosphate stores are used, the body utilizes two different metabolic ways to generate the ATP necessary for performing work. One pathway, aerobic metabolism, requires an adequate supply of oxygen via respiration and circulation to metabolize the metabolic substrates glycogen and glucose and free fatty acids into ATP, carbon dioxide and water.

Anaerobic respiration is the second major metabolic pathway and occurs when the muscle has insufficient oxygen to metabolize substrates to ATP. In the absence of oxygen, the muscle begins to metabolize glycogen stores, producing lactate and hydrogen ions are formed as byproducts. Generally speaking, the circulation will remove lactic acid from the muscle as it is produced. If, however, the work task increases in intensity or the task endures for a long period of time, lactic acid will eventually begin to build up the bloodstream, and hydrogen ions will begin to accumulate in the muscle. As exercise continues or intensity increases a gradual increase in lactic acid and hydrogen ions in the muscle will inhibit energy production and muscle contraction. The point at which lactic acid starts to accumulate in the blood stream is referred to as the lactic acid threshold or the anaerobic threshold (AT).

It is believed the buildup of lactic acid in the muscle may cause premature muscle fatigue and may contribute to poor performance. Thus, AT is an important indicator of performance during exercise and training. Unfortunately, the only current way to measure and monitor directly AT is to sample the blood of a subject undergoing work task and monitor levels of lactic acid. Obviously, withdrawing blood from a subject is not convenient during exercise and it can be time-consuming and uncomfortable. Other ways include measuring the concentration of CO₂ production in the subject that comprise the use of expensive and cumbersome gas exchange equipment. Accordingly, there is a need in the art for methods of determining or approximating the AT in subject undergoing a work task that is non-invasive and mobile.

### SUMMARY OF THE INVENTION

The invention relates to methods for determining one or more metabolic transition points in a subject during a work task. The methods comprise determining the respiration rate (RR) and heart rate (HR) in the subject that is undergoing the work task and calculating the ratio of RR/HR at more than one time point during the work task. The metabolic transition points are identifiable points in time of the RR/HR ratio in the subject that is undergoing a work task.

The invention also relates to systems for determining one or more metabolic transition points in a subject during a work task. The systems comprise means determining the respiration rate (RR) and heart rate (HR) in the subject that is undergoing the work task and means calculating the ratio of RR/HR at more than one time point during the work task. The metabolic transition points are identifiable points in time of the RR/HR ratio in the subject that is undergoing a work task.

### BRIEF DESCRIPTION OF THE DRAWINGS

FIG. 1 depicts the respiratory rate and the heart rate during the exercise. The lower graph of FIG. 1 represents a curve plot of the RR/HR values blue line and the dRR/dHR values. The long arrow pointing to the low point of the curve indicates the AT of this particular subject. At the AT point, the heart rate was 106 beats per minute and the respiratory rate was 15 breaths per minute. The RCP is also indicated. At the RCP, the heart rate rose gradually to 138 beats per minute and the respiratory rate rose to 23 breaths per minute.

### DETAILED DESCRIPTION OF THE INVENTION

The invention relates to methods for determining one or more metabolic transition points in a subject during a work task. The phrase “metabolic transition point,” and variants thereof, is intended to mean points when a subject either changes types of metabolism or compensates for cardiopulmonary parameter during a work task. Examples of metabolic transition points include, but are not limited to, the transition from aerobic oxidation of substrates, such as glycogen or other carbohydrates and fatty acids to anaerobic metabolism of carbohydrates. The point in time at the transition from aerobic metabolism to anaerobic metabolism is often referred to as the anaerobic threshold (AT). The AT point is, generally speaking, the point at which lactic acid begins to accumulate in the blood of the subject because the body produces lactic acid faster than it can be removed from circulation. Of course, having the ability to identify metabolic transition points will also provide the ability to identify the types metabolism in which the subject is, during the work task. For example, identifying the metabolic transition point from aerobic to anaerobic metabolism will also identify the period of aerobic metabolism (AM) and the period of anaerobic metabolism in the subject. Additional examples of metabolic transition points also include, but are not limited to, anaerobic hydrolysis of creatine phosphate, as well as the respiratory compensation points (RCP) in the subject.

In one specific embodiment, the present invention provides methods of determining AT or RCP. In another specific embodiment, the present invention provides methods of determining both the AT and the RCP.

As used herein, “work task” and “work” are used interchangeably and are used as they are in the art. Namely, “work” is the quantity of force that operates on a mass that causes the mass to change location. Exercise, which is physical performance or activity generally directed towards increasing fitness level, is a form of work and is considered to be an example of a “work task” for the purposes of the present invention. Thus, a subject can undergo a work task, for the purposes of the present invention, even though a mass may not change location. For example, a subject running on a treadmill or cycling on a stationary bike is, for the purposes of the present invention, undergoing a work task. Other examples of subjects performing work tasks associated with common exercises and exercise machines will be readily apparent to one of skill in the art.

In one embodiment, the methods comprise determining the respiration rate (RR) and heart rate (HR) in the subject that is undergoing the work task. As used herein, respiration rate is the rate of expiration and inhalation of the subject and is not measured by gas exchange. Indeed, one aspect of novelty of the present application is the ability to determine metabolic transition points in a subject without the need to detect gas exchange. Simply put, RR is the number of breaths a subject takes, per unit time.

Determining the RR in a subject can be accomplished by any means capable of providing the rate. For example, a piezoelectric sensor can be used in a chest belt to measure changes in chest circumference associated with respiration. Piezoelectric sensors used to measure respiratory rate are commercially available (GeneQ, Inc., Montreal, Quebec, CANADA). Another example of a means of measuring respiratory rate includes, but is not limited to, chest belts that comprise other sensing means, such as positional detector sensors that detect movement and electro-conductive elastomeric means, as disclosed in U.S. Pat. No. 4,966,155, which is incorporated by reference. Additional means for determining respiratory rate include such devices as a plethysmograph or the means disclosed in U.S. Pat. No. 5,159,935, which also incorporated by reference. Still another example of an apparatus that can be used to determine RR or dRR is a Respiratory Effort Transducer (BioPac Systems, Goleta, Calif., USA). In one specific embodiment, the RR is determined using at least one piezoelectric sensor. Of course, the methods of the present invention also include manually counting the breaths in a subject per unit time. Other means of determining respiration rate are also contemplated, including, but not limited to, interpolating ECG data or pulse data to determine a RR.

Of course, RR may be determined by measuring gas exchange as well, provided the measurement of gas exchange is capable of providing the respiratory rate of the subject. Means of measuring gas exchange in a subject are well known in the art. Examples of systems that can be used to measure RR, via gas exchange, include, but are not limited to, systems utilizing mixing chambers, breath-by-breath measurements, respired gas volume measurements, flow rate measurements and gas analyzers. Example of an apparatus used to measure gas exchange or gas flow in and out of the lungs include, but are not limited to, the apparatus of U.S. Pat. No. 7,108,659 (is incorporated by reference), or a pneomotachograph, respectively. Additional systems for measuring gas exchange are commercially available.

Determining the heart rate HR of a subject can be accomplished by any means capable of proving the heart rate. Examples of means and apparatuses useful for measuring HR are well known in the art and include, but are not limited to devices with electrocardiogram (ECG) sensors. Heart rate monitors are commercially available. Of course, the HR may also be determined manually, by, for example, placing fingers on any part of the body close to a surface artery or using a stethoscope. In one specific embodiment, the HR is determined using an apparatus or device comprising at least one ECG sensor.

In one embodiment, the methods comprise calculating the ratio of RR:HR at more than one time point during the work task. In one particular embodiment, the RR:HR ratio is calculated at least one time per minute. In another particular embodiment the RR:HR ratio is calculated at least 2, 3, 4, 5, 6, 7, 8, 9 or 10 times per minute. In still another embodiment, the RR:HR ratio is calculated at least once per second. In yet another embodiment, the RR:HR ratio is calculated at least 2, 3, 4, 5, 6, 7, 8, 9 or 10 times per second. In still yet another embodiment, the RR:HR ratio is calculated more than 10 times per second.

In another embodiment, the methods comprise determining the change in respiration rate per unit time (dRR) and the change in heart rate per unit time (dHR) at more than one time point during the work task. Methods and means of determining RR and HR can also be used to determine dRR and dHR. Thus, in one embodiment, the methods comprise calculating the ratio of dRR:dHR at more than one time point during the work task. In one particular embodiment, the dRR:dHR ratio is calculated at least one time per minute. In another particular embodiment the dRR:dHR ratio is calculated at least 2, 3, 4, 5, 6, 7, 8, 9 or 10 times per minute. In still another embodiment, the dRR:dHR ratio is calculated at least once per second. In yet another embodiment, the dRR:dHR ratio is calculated at least 2, 3, 4, 5, 6, 7, 8, 9 or 10 times per second. In still yet another embodiment, the dRR:dHR ratio is calculated more than 10 times per second.

Once the RR:HR ratio or dRR:dHR ratio is determined at each time point, the value of each calculation can be recorded. In one embodiment, the RR:HR ratio values or dRR:dHR ratio values are plotted over time to generate a curve. Examples of methods used to plot curves include, but are not limited to, plotting the data by hand, or inputting the data into curve plotting software. Curve plotting software is commercially available and is even available on many hand-held calculators. Example of curve plotting software include, but are not limited to, SAS (Cary, N.C., USA), SigmaPlot (Systat, San Jose, Calif., USA), MatLab (The MathWorks, Inc. Natick, Mass., USA) and even Excel (Microsoft, Redmond, Wash., USA). The invention should not be limited by the means used to plot the curve, if one is plotted.

In another embodiment, the RR:HR ratio values or dRR:dHR ratio values are recorded onto a recordable medium. Examples of recordable media include, but are not limited to, paper, computer memory and audio recording devices. Additional examples of recordable media should be readily apparent to one of skill in the art.

Once the RR:HR ratio values or dRR:dHR ratio values are plotted or recorded such that each value can be compared to the other values, distinct points in the plot or values can then be assessed. In one embodiment, the metabolic transition points are identifiable points in time of the RR:HR ratio or the dRR:dHR ratio in the subject that is undergoing a work task. In one specific embodiment, the plotted curve of RR:HR or dRR/dHR over time will take a general parabolic shape, and the lowest (smallest) point on the curve will be the point at which the subject transitions to anaerobic metabolism, i.e., the subject has reached AT. In a similar manner, if the data are not plotted, the AT point will be the lowest value of the data points during the work task.

Other metabolic transition points can also be ascertained from the recorded or plotted data. For example, the point at which there is a sharp increase in the slope of the curve (RR:HR or dRR/dHR), after passing the AT, is indicative that the subject has reached the respiratory compensation point. To be clear, the AT, RCP and other metabolic transition points are reported in terms of the subjects hear rate.

By determining the AT of a subject in this way, additional information can be gleaned from the subject, including, but not limited to determining the subject's substrate use during the work task and determining the fitness level of the subject. For example, the respiratory quotient (RQ) for a person at rest is about 0.7, which indicates the oxidation of fat, rather than carbohydrates or proteins. At about 60% of maximal aerobic exercise, the RQ increases to about 1.0, indicating the switch from fat metabolism to primarily carbohydrate metabolism. At the RCP, the RQ is greater than 1.0 indicating pure carbohydrate metabolism. Accordingly, knowing the status of the subject in relation to the heart rate and metabolic transition points, it is possible for the subject to determine substrate usage during the work task.

The invention also relates to systems for determining one or more metabolic transition points in a subject during a work task. The systems comprise means determining RR and HR or dRR and dHR in the subject that is undergoing the work task and a means for calculating the ratio of RR:HR or dRR:dHR at more than one time point during the work task. Examples of systems include, but are not limited to, those systems disclosed in U.S. Pat. Nos. 6,405,077; 6,411,841; 6,540,686, 6,687,535 and United States Pre-Grant Publication No. 2006/0004265, all of which are incorporated by reference. Other examples of systems are disclosed in PCT Publication No. WO 2004/016173, which is incorporated by reference.

In one embodiment, the systems of the present invention comprise a computer processing unit. The processor controls the operation of the device and also provides control of various functionalities and output of the system. The processor can be a central processor that controls functionality via a bus structure or other communications interface. The processor can also be implemented by distributing the processing functions among one or more of the various components utilized to implement the functionalities of the devices. The computer processing unit can be any form, provided that the computer processing unit is capable of generating the desired data. For example, the computer processing unit may be within a personal computer, or a wrist watch or a mobile telephone, such as the system disclosed in United States Pre-grant Publication No. 2006/0063980, which is incorporated by reference.

One component of the computer processing unit will include memory. Memory is used to provide storage for program data or other data used by computer processing unit during operation and can be implemented using various RAM or ROM memory devices. Memory can be used for example, to store operating instructions and to provide memory registers for operating and storage.

Memory can also be used in conjunction with a storage device such as, but not limited to, a disk storage device or a flash memory device. A storage device can also be used to store program instructions, control and generate curves, operational data, history logs, and other data such as user input data which may be desired to be stored within the device. Alternatively, the storage device, if one is present, need not be within the device. In one embodiment, the storage device will not store large amounts of data; but the data or instructions it stores is capable of being accessed frequently and rapidly. In another embodiment, a cache is present to minimize latencies associated with retrieving frequently used data or instructions from the storage device. In more specific embodiments, the storage device may store less 1 gigabyte (GB), less than 500 megabytes (MB), less than 250 MB, less than 100 MB, less than 50 MB, less than 20 MB, less than 10 MB, less than 9 MB, less than 8 MB, less than 7 MB, less than 6 MB, less than 5 MB, less than 4 MB, less than 3 MB, less than 2 MB or less than 1 MB of data and/or instructions. In another specific embodiment, the storage device may store large amounts of data, for example 1 GB or more of data.

The memory of the device will comprise machine executable instructions. The machine executable instructions control the input and storage of data and recordation of data within the device. The end-user can select and input into the device specific data, such as, but not limited to gender, weight, height, fitness level and age and parameters to be measured, i.e., RR, HR, dRR, dHR, and the machine executable instructions will record the selected parameters.

The computer, memory and machine executable instructions should be in informational connectivity with the system components that are responsible for measuring the selected parameters. A used herein, informational connectivity is used to indicate that information collected and/or recorded in the various components of the system can be transmitted to another component of the system at some point. The connectivity can, but need not, be simultaneous with the detection and/or recordation of various parameters. Thus, in one embodiment, the system comprises a memory that records the selected parameters (RR, HR, dRR, dHR) during the work task. At a later time, the recorded history of the parameters is transmitted to a computer capable of generating a curve or analyzing the data to determine the AT, or some other metabolic transition point, after the user has completed the work task. In another embodiment, the detected parameters are transmitted to a computer capable of generating a curve or analyzing the data in real time, to provide the user with real time data regarding the AT, or some other metabolic transition point.

In another embodiment, the devices of the present invention may comprise one or more communications ports. The communications port(s) is (are) capable of connecting to another device such as, but not limited to, a computer, a disk drive, a flash memory drive, a monitor, a printer or other similar device. For example, the functions of the device can be updated or altered, and the device can be calibrated or recalibrated with new machine executable instructions from a computer, CD or DVD via the communications port. The devices are not limited by the types of communications ports. Examples of communications ports include but are not limited to universal serial bus (USB), an audio/video serial bus (IEEE 1394) (“firewire”), and infrared port and a radio frequency port. Radio ports include Bluetooth® ports, Wi-Fi ports and the like.

Thus, in one embodiment of the present invention, the system comprises a transmitter that transmits information from one component to another component of the system. For example, a chest belt may comprise one or more transmitters for transmitting RR information and/or HR information to the computer processing unit, such that the computer processing unit can manipulate the incoming data to determine the AT or other metabolic transition points.

A component of the systems of the present invention may also comprise a user interface. Various user interfaces can be provided to facilitate user control and to enhance operability of the devices. Input interfaces include, but are not limited to, data entry devices such as a keyboard, keypad, touch-screen display, mouse, voice recognition input, or other data entry device. In one specific embodiment, the user interface comprises a non-numerical keypad. Output interfaces include, but are not limited to, a display screen, monitor, a printer, a speaker or other output device. In another specific embodiment, the user interface comprises a display screen. In yet another specific embodiment, the user interface comprises both a non-numerical keypad and a display screen.

The devices of the present invention may optionally comprise an internal power source, used to power the various components of the device. In one embodiment, the power source is rechargeable. In another embodiment, the internal power source is not rechargeable.

In another embodiment of the present invention, the end-user can access a database or other program through the internet to record personalized data and develop an individual training regimen, based upon the calculated AT or other metabolic compensation points. One example of an online embodiment is disclosed in United States Pre-Grant Publication No. 2006/0250524, which is incorporated by reference.

The methods and systems of the present invention may also be used to diagnose or screen for abnormal cardiovascular conditions in a subject. The methods comprise determining the AT, RCP or other metabolic transition points in a subject and comparing the determined values against normal values of the AT, RCP or other measured metabolic transition point and determining a difference between the measured values of the normal values. A difference between the measured values and the normal values may be indicative of a cardiovascular condition.

As used herein the term cardiovascular condition includes, but is not limited to, coronary artery disease, high blood pressure, pulmonary hypertension, cardiac function, tachycardia, heart failure, heart valve disease, severity of peripheral vascular disease, integrity of certain autonomic nervous system reflexes (including the carotid-baro reflex and the vagovagal reflex), intracardiac shunting of blood, and the like.

As used herein, the term “diagnose” means to confirm the results of other tests or to simply confirm suspicions that the subject may have an abnormal condition. A “test,” on the other hand, is used to indicate a screening method where the patient or the healthcare provider has no indication that the patient may, in fact, have an abnormal condition and may also be used to assess a patient's likelihood or probability of developing a disease or condition in the future. The methods of the present invention, therefore, may be used for diagnostic or screening purposes. Both diagnostic and testing can be used to “stage” the obese condition in a patient. As used herein, the term “stage” is used to indicate that the abnormal condition can be categorized, either arbitrarily or rationally, into distinct degrees of severity. The categorization may be based upon any quantitative characteristic that can be separated, or it may be based upon qualitative characteristics that can be separated. The term “stage” may or may not involve disease progression.

“Normal values” of a given value may be assessed by measuring levels of the AT, RCP or other metabolic transition point in a known healthy subject, including the same subject that is later screened or being diagnosed. Normal levels may also be assessed over a population sample, where a population sample is intended to mean either multiple measurements from a single subject or at least one measurement from a multiple of subjects. Normal values, in terms of a population of samples, may or may not be categorized according to characteristics of the population including, but not limited to, sex, age, weight, BMI, ethnicity, geographic location, fasting state, state of pregnancy or post-pregnancy, menstrual cycle, general health of the patient, alcohol or drug consumption, caffeine or nicotine intake and circadian rhythms.

### EXAMPLES

### Example 1

### Comparison of the Methods of the Present Invention to CPET Methods

Each subject in the experiment performed a physician-supervised, standard, progressively increasing work task on an electromagnetically braked cycle ergometer. Four healthy males, age 40-60, were chosen to perform the tests. Measurements were made during 3 minutes of rest, 2 minutes of unloaded leg cycling at 60 rpm followed by a progressively increasing work task exercise of 10 watts/min to maximum tolerance, and 2 minutes of recovery.

To determine AT using the methods of the present invention, the subjects were also connected to a piezoelectric sensor (Pneumotrace II from GeneQ) to measure respiration rate. In addition, the subjects were fitted with heart rate monitors with a 3 lead ECG (Biopac Systems). The heart rate monitor and the piezoelectric sensor were connected to a computer via an analog to digital (A/D) card. (National instruments, Austin, Tex., USA). Respiratory rate and heart rate were recorded continuously and the following parameters were analyzed: RR/HR and dRR:dHR. Data was recorded and calculated on a breath by breath basis, interpolated per second and averaged over 10-second intervals.

To determine the AT using standard CEPT equipment, a gas exchange apparatus (Medical Graphics, St. Paul, Minn., USA) was used to measure concentrations of CO₂ and O₂ in expired gas from the individuals. In addition, a 12-lead ECG was used to measure HR and blood pressure was monitored and recorded using a cuff. The CPET system also measured the saturation of peripheral (arterial) oxygen (SpO₂), minute ventilation (VE), O₂ uptake (VO₂), CO₂ output (VCO₂) and other exercise variables were computer-calculated breath by breath, interpolated per second and averaged over 10-second intervals.

Data analysis was performed using C++ language based on a .Net framework. Using the CPET methods, the anaerobic threshold was defined as the beginning of the increase in the VE/VO₂ without an increase in the VE/VCO₂ during the increasing work load, which is a commonly accepted definition in the art. Using the methods of the present invention, At was determined as the lowest value of the RR/HR during the exercise period. The results of the comparison of the 4 subjects are presented in Table 1, below.

TABLE 1
Comparison of CPET Methods to the Methods of the Invention

| Subject | CPET | RR/HR |
|---|---|---|
| 1 | 110 | 110 |
| 2 | 104 | 106 |
| 3 | 110 | 110 |
| 4 | 86 | 86 |

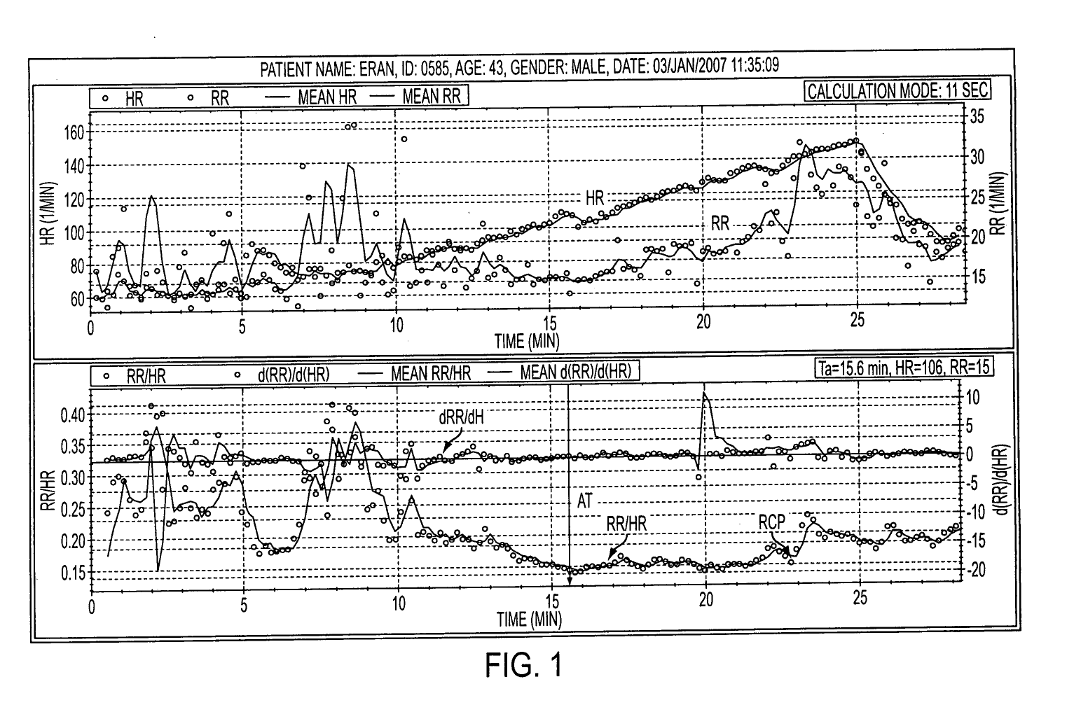

The results of the test are also shown in FIG. 1. The upper graph of FIG. 1 represents respiratory rate and the heart rate during the exercise. The lower graph of FIG. 1 represents a curve plot of the RR/HR values blue line and the dRR/dHR values. The long arrow pointing to the low point of the curve indicates the AT of this particular subject. At the AT point, the heart rate was 106 beats per minute and the respiratory rate was 15 breaths per minute. The RCP is also indicated. At the RCP, the heart rate rose gradually to 138 beats per minute and the respiratory rate rose to 23 breaths per minute. In the same subject, the AT was determined to be 105 beats per minute and the respiratory rate was 15 breaths per minute, using the CPET methods (data not shown).

### Example 2

### Determination of Fitness Level Using the Generated Data

Using the data generated in the above example, and inputting subject specific data, such as male, age: 43, weight: 73 kg, height: 170 cm, a general fitness level was determined for subject 2 from the Example 1.

TABLE 2
Fitness and Personal Data of Subject 2

| | Heart rate | Respiration rate | % predicted |
|---|---|---|---|
| Rest | 58 | 12 | |
| AT (45%) | 106 | 15 | 110% |
| Max | 165 (pred) | 30 | 105% |

### Example 3

### Determination of Metabolite Usage Using the Generated Data

Based upon the data generated herein, one can calculate metabolic substrate usage during exercise. Metabolic substrate usage was determined according to heart rate and burned calories per min according to a subject's body weight.

TABLE 3
Metabolic Substrate Usage

| Substrate | Heart rate | %* | Calories per min |
|---|---|---|---|
| Fat | 60-100 | 85% | 5 |
| Fat | 100-115 | 50% | 6.5 |
| Carbohydrates | 100-1115 | 50% | 7.8 |
| Carbohydrates | 110-125 | 85% | 10 |
| Carbohydrates | 125 maximal | 100% | Max 15 |

*The percentage column indicates percentage from the total calories burned. For example, in row 1, fat constituted 85% of the total calories burned at that point.

### Example 4

### System

The following is just one example of the capabilities of the system comprising machine executable instructions (MEIs).

First a transmitter transmits data, either wirelessly or hard-wired from the sensor to the computer housing the MEIs. The MEIs will then filter the data by comparing sensor data to 3 previous samples. Any parameter that is 20% higher or lower will be excluded. The MEIs will then calculate each respiratory cycle (inspiration and expiration) to determine the RR, and the MEIs will also determine HR from the transmitted ECG information. In one embodiment, data is continuously transmitted to the computer housing the MEIs and the MEIs continuously record and/or generate data.

The MEIs will then plot the data over time or compared changes in the RR:HR ratio. The lowest point on the curve, or the lowest value of the RR/HR ratio is determined as the AT. In one embodiment, data outside of the range of 25%-90% of the heart rate reserve is excluded from the calculation. As is used in the art, the heart rate reserve is the difference between a subject's measured or predicted maximum heart rate and their resting heart rate.

The MEIs will also determine the respiratory compensation point after measuring the AT, based upon a sudden up-slope of the curve or a sudden increase in the value of the RR:HR ratio In one embodiment, data outside of the range of less than 50% heart rate reserve is excluded from the calculation.

The system will then display the changes of RR/HR in a graph or in a table and highlights the calculated AT and/or the RCP.

The system may also be capable of providing a visual and/or auditory signal or alarm when the RR:HR ratio is the lowest during patients exercise, i.e., when the subject reaches the AT and also when RR:HR ratio increases dramatically, i.e., when the subject reaches the RCP. Of course, the MEIs may be programmed to provide an alarm at virtually any time during the work task. For example, an alarm may be programmed for instances when there are no detectable changes in the transmitted data over a pre-determined period of time, i.e., the subject has reached a steady state. The alarm may, in turn, prompt the user to increase or decrease exercise intensity. In another example, an alarm may sound when the HR drops below RCP or AT during the recovery period.

The system may also be capable of analyzing the data to provide gives information about metabolic substrate utilization, e.g., carbohydrates, lipids, according to the RR:HR data.

The MEIs may also calculate the total calories burned during the work task in accordance with the subject's weight, age, gender and HR or RR. In addition, the MEIs may calculate the type of exercise during the work task, such as, but not limited to, aerobic exercise, cardiopulmonary exercise, fat burning exercise and interval training.

The MEIs may also estimate the subject's fitness level according to the determined AT in relation to the to maximal heart rate during exercise. In normal and healthy subjects, for example, AT may occur at about 50% of the maximum predicted heart rate. Well-trained athletes, on the other hand, AT may occur at about 85% of the maximum predicted heart rate. In contrast, subjects that are considered unfit or with heart problems may experience AT at about 30-35% of the predicted maximum heart rate. This information can be used by the subject to improve fitness levels and develop a training regimen.

1. A method for determining one or more metabolic transition points in a subject during a work task, said method comprising
a) determining the respiration rate (RR) or any other related parameter in the subject that is undergoing the work task;
b) determining the heart rate (HR) of the subject undergoing the work task;
c) calculating the ratio of RR/HR at more than one time point during the work task; and
d) identifying at least one distinct point in time of the RR/HR ratio,
wherein the identified distinct point(s) correlates with the metabolic transition point of the subject undergoing a work task.

2. The method of claim 1, wherein at least two distinct points in time are identified.

3. The method of claim 2, wherein the metabolic transition points are selected from the group consisting of aerobic metabolism (AM), anaerobic threshold (AT) and respiratory compensation point (RCP).

4. The method of claim 3, wherein AT or RCP are determined.

5. The method of claim 4, wherein the ratio of RR/HR is calculated at least once per minute during the work task.

6. The method of claim 5, wherein the ratio is calculated at least 5 times per minute during the work task.

7. The method of claim 6, wherein identifying at least one distinct point comprises plotting a curve of the RR/HR ratio versus time and identifying specific points along the plotted curve.

8. The method of claim 7, wherein the curve plotting is performed by a computer.

9. The method of claim 7, wherein the curve plotting is performed by hand.

10. The method of claim 9, wherein the AT is determined.

11. The method of claim 10, wherein the AT is the lowest point on the plotted curve.

12. The method of claim 6, wherein identifying at least one distinct point comprises recording each value of the RR/HR ratio onto recordable medium and identifying the specific values of the ratio in relation to all recorded ratio values.

13. The method of claim 12, wherein the AT is determined.

14. The method of claim 13, wherein the AT is the lowest ratio value in relation to all recorded ratio values.

15. A system for determining one or more metabolic transition points in a subject during a work task, said system comprising:
a) means for determining the respiration rate (RR) or any other related parameter in the subject that is undergoing the work task;
b) means for determining the heart rate (HR) of the subject undergoing the work task;
c) means for calculating the ratio of RR/HR at more than one time point during the work task; and
d) means for identifying at least one distinct point in time of the RR/HR ratio,
wherein the identified distinct points correlate with the metabolic transition point of the subject undergoing a work task.

16. The system of claim 15, wherein the system is capable of identifying at least two distinct points in time.

17. The system of claim 16, wherein the metabolic transition points are selected from the group consisting of aerobic metabolism (AM), anaerobic threshold (AT) and respiratory compensation point (RCP).

18. The system of claim 17, wherein AT or RCP are determined.

19. The system of claim 18, wherein the ratio of RR/HR is calculated at least once per minute during the work task.

20. The system of claim 19, wherein the ratio is calculated at least 5 times per minute during the work task.

21. The system of claim 15, further comprises a memory capable of storing the values of the RR/HR ratio.

22. The system of claim 21, further comprising a computer processing unit which processes machine executable instructions.

23. The system of claim 22, wherein the machine executable instructions, when executed by the computer processing unit, cause the system to identify the at least one distinct point.

24. The system of claim 23, wherein the machine executable instructions, when executed by the computer processing unit, cause the system to generate a curve of the RR/HR ratio versus time and to identify specific points along the generated curve.

25. The system of claim 24, wherein the AT is determined.

26. The system of claim 25, wherein the AT is the lowest point on the plotted curve.

27. The system of claim 20, herein identifying at least one distinct point comprises recording each value of the RR/HR ratio onto recordable medium and identifying the specific values of the ratio in relation to all recorded ratio values.

28. The system of claim 27 wherein the AT is determined.

29. The system of claim 28 wherein the AT is the lowest ratio value in relation to all recorded ratio values.

30. The system of claim 15, wherein said means for determining RR comprises at least one piezoelectric sensor or plethysmograph.

31. The system of claim 15, wherein said means for determining HR comprises at least one an electrocardiogram (ECG) sensor.

32. The system of claim 15, further comprising a graphical user interface (GUI).

33. The system of claim 32 wherein the GUI allows the subject to input data for at least one value selected from the group consisting of age, weight, gender, training goals and target training zones.

34. A method for determining one or more metabolic transition points in a subject during a work task, said method comprising
a) determining the change in respiration rate per unit time (dRR) in the subject that is undergoing the work task;
b) determining the change in heart rate per unit time (dHR) of the subject undergoing the work task;
c) calculating the ratio of dRR/dHR at more than one time point during the work task; and
d) identifying at least one distinct point in time of the dRR/dHR ratio,
wherein the identified distinct points correlate with the metabolic transition point of the subject undergoing a work task.

35. The method of claim 34 wherein at least two distinct points in time are identified.

36. The method of claim 35 wherein the metabolic transition points are selected from the group consisting of aerobic metabolism (AM), anaerobic threshold (AT) and respiratory compensation point (RCP).

37. A method of diagnosing a cardiovascular abnormality, said method comprising
a) obtaining the value of at least one of a subject's metabolic transition points; and
b) comparing the obtained subject's metabolic transition point to values of metabolic transition points in a normal population,
wherein a difference in the obtained value and the normal value is indicative of a cardiovascular abnormality in the subject.

38. The method of claim 37, wherein the abnormal cardiovascular condition is selected from the group consisting of: such as coronary artery disease, high blood pressure, pulmonary hypertension and cardiac function.

39. The method of claim 1, wherein the RR or the any other related parameter is determined by the rate of expiration and inhalation of the subject.

40. The system of claim 15, wherein the RR or the any other related parameter is determined by the rate of expiration and inhalation of the subject.

41. The method of claim 1, wherein the method further comprises measuring expiration and inhalation prior to determining the RR or the any other related parameter.

42. The system of claim 15, wherein the system further comprises means for measuring expiration and inhalation prior to determining the RR or the any other related parameter.
